(19) United States
(12) Patent Application Publication
(10) Pub. No.: US 2019/0380804 A1
(43) Pub. Date: Dec. 19, 2019

(54) WARMING SYSTEM FOR MEDICAL EQUIPMENT

(71) Applicant: BATRIK MEDICAL MANUFACTURING INC., Dorval (CA)

(72) Inventor: Mitchell K. GOLDBERG, Westmount (CA)

(21) Appl. No.: 16/436,611

(22) Filed: Jun. 10, 2019

Related U.S. Application Data
(60) Provisional application No. 62/685,693, filed on Jun. 15, 2018.

(51) Int. Cl. A61B 50/00; F24F 3/16; H05B 1/02

(52) U.S. Cl. CPC A61B 50/00; F24F 3/161; A61B 2050/105; A61B 2050/0018; H05B 1/025

(57) ABSTRACT

The present technology generally relates to warming systems for warming medical equipment. The present technology also generally relates to methods for warming medical equipment using the warming systems of the present technology.

### CROSS-REFERENCE TO RELATED APPLICATION

This application claims the benefit of U.S. Provisional Application No. 62/685,693, filed Jun. 15, 2018, the disclosure of both of which is incorporated herein by reference in its entirety.

### FIELD OF TECHNOLOGY

The present technology generally relates to warming systems for warming medical equipment. The present technology also generally relates to methods for warming medical equipment using the warming systems of the present technology.

### BACKGROUND INFORMATION

Various types of medical items require to be heated to specific temperatures prior to utilization in medical procedures. Generally, medical items may be heated for limited time intervals to preserve their effectiveness. These medical items typically include items such as intravenous solutions, irrigation fluids, surgical instruments, bottles and blankets. Intravenous (IV) solutions are typically stored in a cool environment and, consequently, require heating to precise temperatures to prevent thermal shock and injury from occurring during infusion of the solution into a vein of a patient.

In order to provide the required heat to the medical items for use thereof in medical settings, warming systems are provided for regulating and/or monitoring temperature of the medical items. For example, warming systems may be disposed within operating rooms to heat medical items to desired temperatures to be used in surgery. Some warming systems are provided with separate warming compartments that may be heated to different temperatures to heat various medical items simultaneously.

One drawback of the current warming systems is that they do not address foreign air flowing into the warming system. When foreign air flows inside of the warming system, such as through an air inlet or or when the door of the warming system is opened, the foreign air comes in contact with medical items contained inside of the warming system. Under such circumstances, the foreign air may adversely alter the temperature of the medical items, and assist in bringing airborne contaminants of adjacent clinical environment into contact with the medical items.

Therefore, there exists a need in the art for warming systems having features that decrease the risk of adverse alterations to the temperature of the medical items placed therein as well as decrease the risk of contamination of such medical equipment.

### SUMMARY OF DISCLOSURE

Other aspects and features of the present disclosure will become apparent to those ordinarily skilled in the art upon review of the following description of specific embodiments in conjunction with the accompanying drawings.

According to many aspects, the present technology relates to a warming system for warming medical items comprising: at least one warming unit for receiving the medical items, wherein the at least one warming unit maintains a desired temperature; a circulation unit for circulating air through the at least one warming unit; and a particulate air filtration unit for removing particles of air passing therethrough, the particulate air filtration unit in fluid communication with the at least one warming unit.

A warming system for warming medical items comprising: a housing including an air inlet, an air outlet and at least one warming compartment, the at least one warming compartment defining an access opening for receiving the medical items therein, wherein the air inlet, the air outlet, the at least one warming compartment and the access opening are in fluid communication; a particulate air filtration unit for removing particles from air flowing therethrough; and at least one warming unit operatively connected to the at least one warming compartment for imparting a desired temperature thereto; and wherein air flowing through the air inlet flows through the particulate air filtration unit before flowing into the at least one warming compartment. In some implementations, the particulate air filtering unit is in fluid communication with the at least one warming compartment. In some implementations, the air inlet is fluidly connectable to a clean air supply system.

Other aspects and features of the present technology will become apparent to those ordinarily skilled in the art upon review of the following description of specific embodiments in conjunction with the accompanying drawings.

### BRIEF DESCRIPTION OF THE FIGURES

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a front side elevation view of a schematic representation of a warming system according to one embodiment of the present technology.

FIG. 2 is a left side elevation view of the warming system as depicted in FIG. 1.

FIG. 3 is a perspective view taken from a front, right side of the warming system as depicted in FIG. 1.

FIG. 4 is a section view of the warming system as depicted in FIG. 2.

FIG. 5 is a close-up view of a top portion of the warming system as depicted in FIG. 4.

FIG. 6 is a schematic representation of a warming system having two warming units according to a further embodiment of the present technology.

FIG. 7 is a schematic representation of circulation and recirculation pathways in a warming system according to a further embodiment of the present technology.

It should also be noted that, unless otherwise explicitly specified herein, the drawings are not to scale.

### DESCRIPTION OF TECHNOLOGY

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant technology. Hence, the following specification is intended to illustrate some particular embodiments of the technology, and not to exhaustively specify all permutations, combinations and variations thereof.

As used herein, the singular form “a” “an” and “the” include plural referents unless the context clearly dictates otherwise.

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first warming unit” and “third warming unit” is not intended to imply any particular order, type, chronology, hierarchy or ranking (for example) of/between the warmer units, nor is their use (by itself) intended imply that any “second warmer unit” must necessarily exist in any given situation. Further, as is discussed herein in other contexts, reference to a “first” element and a “second” element does not preclude the two elements from being the same actual real-world element. Thus, for example, in some instances, a “first warming unit” and a “second warming unit” may be a same “warming unit” or both have a portion of a same warmer unit in common. In other cases, they may be different “warming units” entirely.

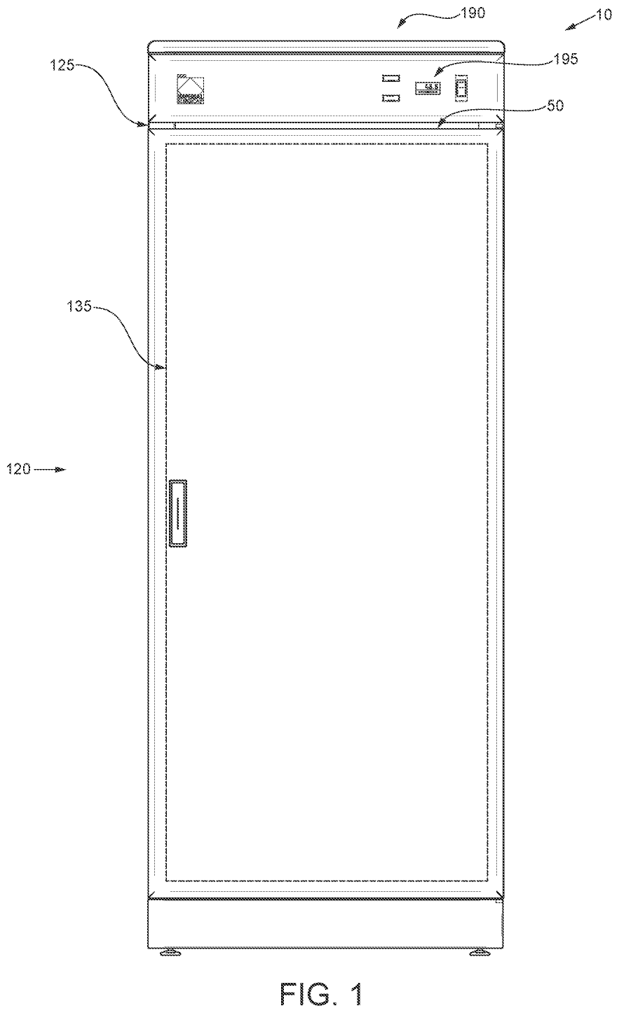

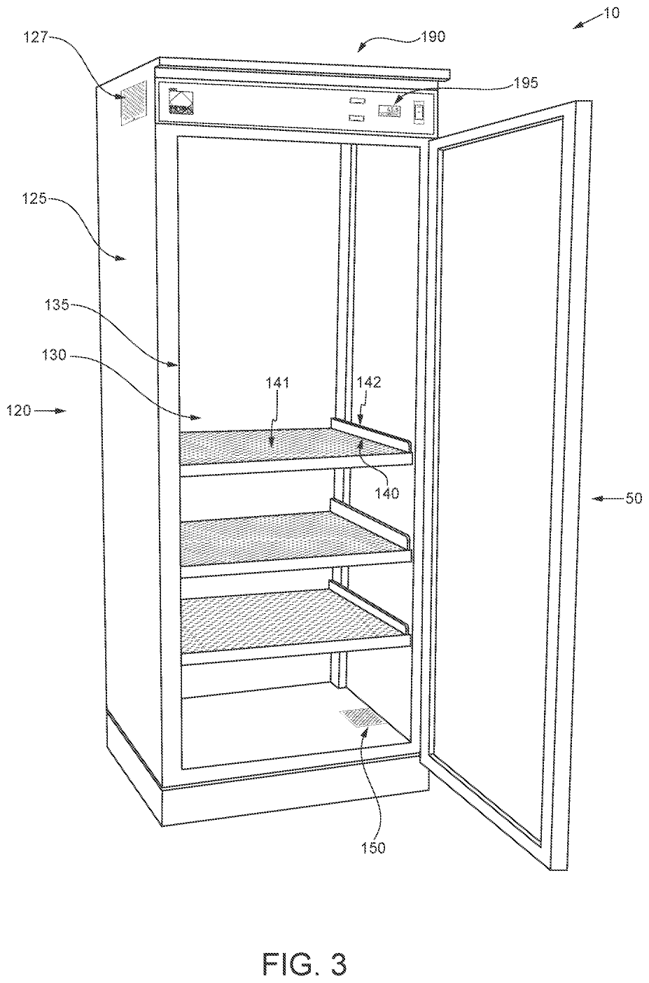

With reference to FIGS. 1 to 5, where all reference numbers refer to alike parts, there is shown a warming system 10 for warming medical items according to a first embodiment of the present disclosure. The warming system 10 comprises a warming unit 120 for heating the medical items (not shown) the warming unit 120 being enclosed in a housing 125. Conventionally, the housing 125 may have exterior and interior portions constructed of materials (e.g. including but not limited to medical grade stainless steel) appropriate for maintenance of aseptic conditions, the interior portion being structured for air-sealed and insulated operation of the warming unit 120. The warming system 10 also comprises a circulation unit 130 and a particulate air filtration unit 140, both in fluid communication with the warming unit 120 for circulation and filtration of the air being circulated into the warming unit 120, respectively. The warming system 10 further comprises a door 50 structured and arranged with respect to the warming unit 120 such that the interior of the warming unit 120 is rendered accessible and in direct fluid communication with the clinical environment when the door 50 is in an open position (such as shown on FIG. 3) and is rendered inaccessible and not in direct fluid communication with the clinical environment when the door 50 is in a closed position (such as shown on FIG. 1).

Warming Unit

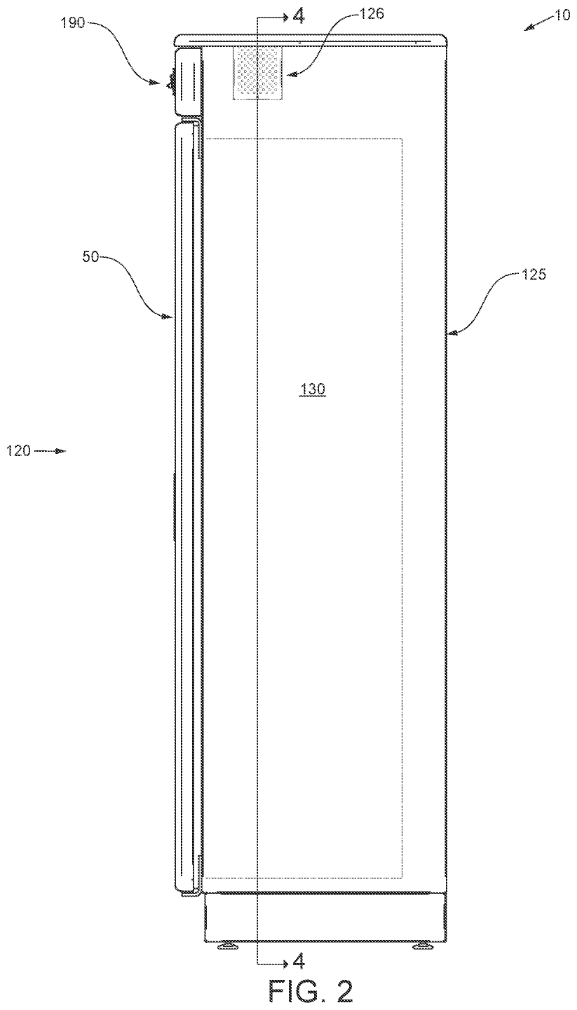

Referring to FIGS. 2 and 3, the warming unit 120 includes a warming compartment 130 located within the housing 125 to receive the medical items. The housing 125 further includes an air inlet 126 and an air outlet 127. The housing 125, the air inlet 126, the warming compartment 130 and the air outlet 127 are in fluid connection with one another. In some implementations, the warming system 10 may be fluidly connectable to a clinical establishment's heating, ventilating and air-conditioning (HVAC) system. For instance, the air inlet 126 and air outlet 127 are fluidly connectable to a clean air supply system (not shown) and a stale air evacuation system (not shown), respectively.

The warming compartment 130 defines an access opening 135 (FIG. 1) for receiving the medical items therethrough. In some implementations of this embodiment, the warming compartment 130 includes one or more shelves 140. In some implementations, the one or more shelves 140 are installed into the warming compartment 130 via supporting elements 142. In some instances, supporting elements 142 may be tracks (FIG. 3), rails, pins, hooks or the like that allow to support the shelves 140 when placed into the warming compartment 130. In some instances, shelves 140 are removable from the warming compartment 130 and out of the warming system 10. In some other instances, the shelves 140 are permanently installed in the warming compartment. In some implementations, the shelves 140 may be placed in the warming compartment 130 in a plurality of positions. Under certain circumstances, some positions may assist in adjusting air flow in the warming compartment 130 and around the medical instruments placed therein. In some implementations, the warming compartment 130 includes at least one shelf 140 that is horizontally displaceable to extend from the access opening 135 when the door is in the open position, thereby facilitating access to the medical items. In some implementations, the one or more shelves 140 comprise a plurality of apertures 141 (such as shown on FIG. 3) that allow air to flow therethrough.

In some other implementations, the warming compartment 130 does not include any shelf 140 so that the warming compartment 130 can accommodate larger medical equipment. In some instances, hooks or other similar elements may be installed on the inner walls of the warming compartment 130 to hang medical devices.

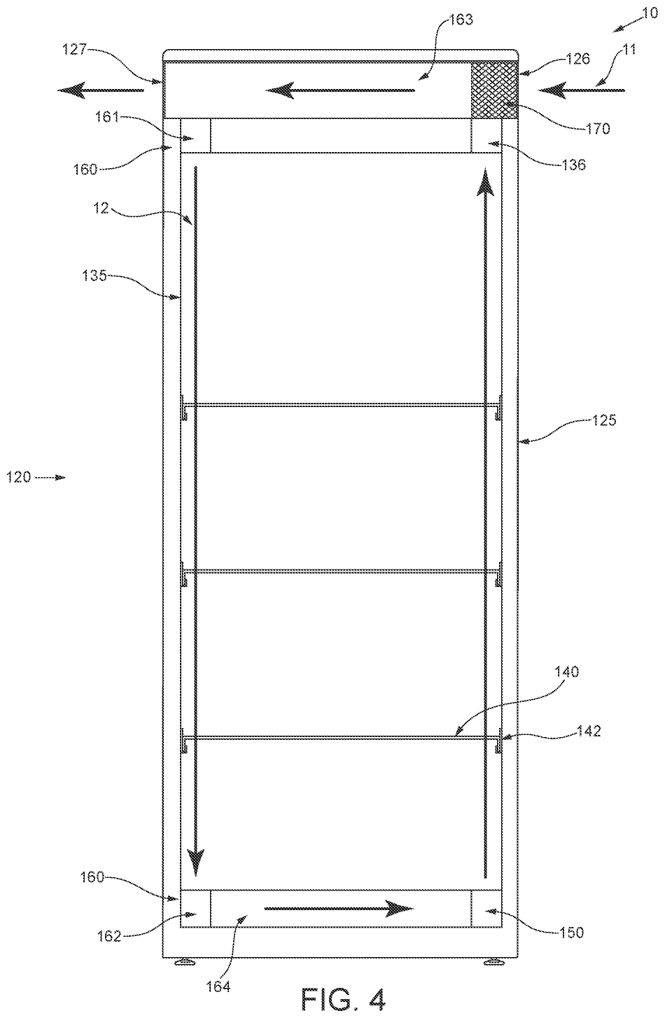

In the present embodiment of the present technology, the warming system 10 includes a heating unit 150 for imparting a desired temperature to the warming compartment 130. As best shown in FIG. 4, the heating unit 150 is located in a lower portion of the housing 125 and fluidly connected to the warming compartment 130. In other embodiments, the heating unit 150 is located in a different portion of the housing 125. In some implementations, the heating unit 150 is structured and arranged to generate heat by converting electricity, and includes one or a combination of metal heating element, ceramic heating element, polymer heating element and composite heating element. In some implementations, heat generated by the heating unit 150 is imparted to the warming compartment 130 by one or a combination of convection, conduction and radiation. In some implementations, the heating unit 150 is a portion of a HVAC system.

The warming unit 120 maintains a desirable temperature and, in some embodiments, the warming unit 120 is heatable up to the desired temperature. The desired temperature may be of any temperature at which it is desirable to warm the medical device to. In some embodiments, the warming system 10 comprises a temperature sensor to measure temperature in the warming compartment 130 of the warming unit 120.

In some implementations, when the warming system 10 is in operation, the warming compartment 130 has an operation temperature ranging from between about 20° C. and about 200° C.; between about 20° C. and about 150° C.; between about 20° C. and about 125° C., or between about 20° C. and about 100° C. In some other embodiments, the warming compartment 130 has an operation temperature ranging from about the temperature of the environment/room temperature to a temperature that is adequate for sterilization of the medical items disposed in the warming compartment 130. In some embodiments, the temperature in the warming compartment 130 is maintained at about 37° C. (about 98° F.). In some embodiment, the at least one warming unit 120 is heatable to a temperature of about 82° C. (about 180 F).

In some implementations, when the warming system 10 is not in operation and has transferred any residual heat resulting from earlier operation, the warming compartment 130 has a temperature that is similar to the temperature of environment/room, and may vary therefrom by no more than ±1° C.; ±2° C.; and ±5° C.

In some implementations, the housing 125 has a temperature that is similar to the temperature of environment/room, and may vary therefrom by no more than ±1° C.; ±2° C.; and ±5° C.

The operation temperature of the warming compartment 130 may depend on the type and quantity of medical items that are to be placed therein, on the intended clinical use of such medical items and on the clinical environment.

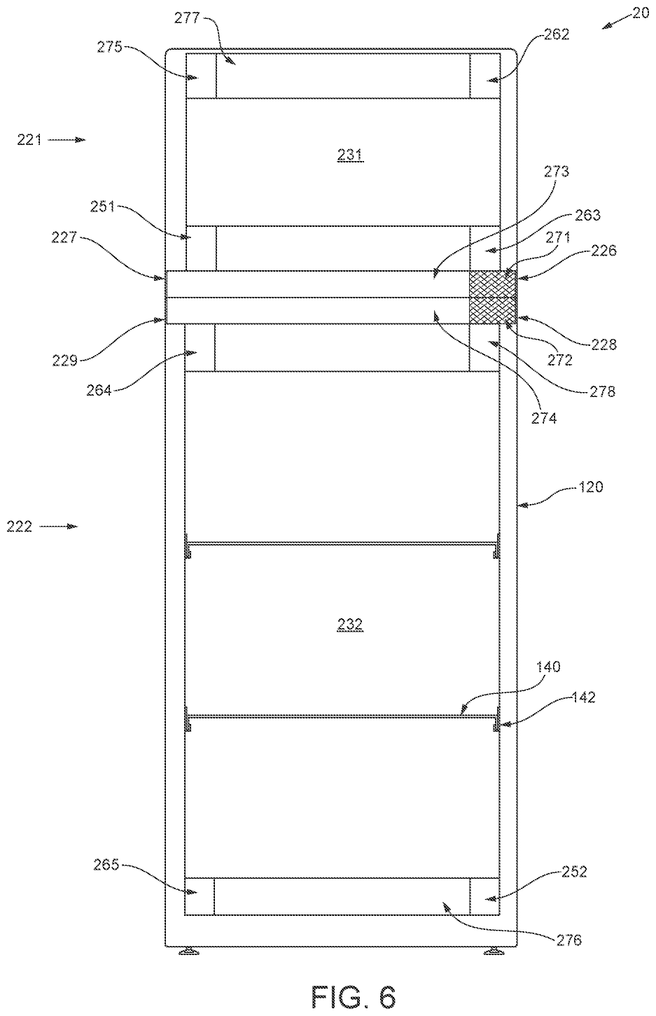

Under certain circumstances, a warming system 20 having a plurality of warming units 120 may be desirable. As shown in FIG. 6, the warming system 20 has an upper warming unit 221 and a lower warming unit 222. In some embodiments, the warming system 20 may have one of 2, 3, 4, and 5 warming units 120. In some instances, these warming units 221, 222 each have their own and separate doors (not shown). In some embodiments, the warming units 221, 222 are heatable to different temperatures.

Circulation Unit

The warming system 10 further includes a circulation unit 160 fluidly connected to the warming unit 120 for generating air flow therein. In some embodiments, the circulation unit 160 includes a ventilation element such as a fan. In the present embodiment, the circulation unit 160 comprises an upper circulation fan 161 located in an upper portion of the housing 125, between an upper circulation cavity 163 and the warming compartment 130. The circulation unit 160 further comprises a lower circulation fan 162 which is located in the lower portion of the housing 125, between the warming compartment 130 and a lower circulation cavity 164. In other embodiments, the circulation unit 160 comprises circulation fans 161, 162 that are located in different portions of the housing 125.

Referring to FIG. 4, the upper circulation unit 161 circulates air downward into the warming compartment 130. The lower circulation unit 162 circulates air upward out of the warming compartment 130. In some embodiments, the circulation unit 160 includes a ventilation element such as a fan (not shown) for directing air flow. In some implementations, the ventilation element has operation parameters (e.g. including, but not limited to duration and speed) that are electronically controllable to achieve a desired air flow. In some implementations, at least one circulation unit 160 may be located elsewhere with respect to the housing 125 (e.g., inside or outside of the housing 125). In some implementations, at least a portion of the circulation unit 160 is a portion of a heating, ventilating and air-conditioning (HVAC) system.

Particulate Air Filtration Unit

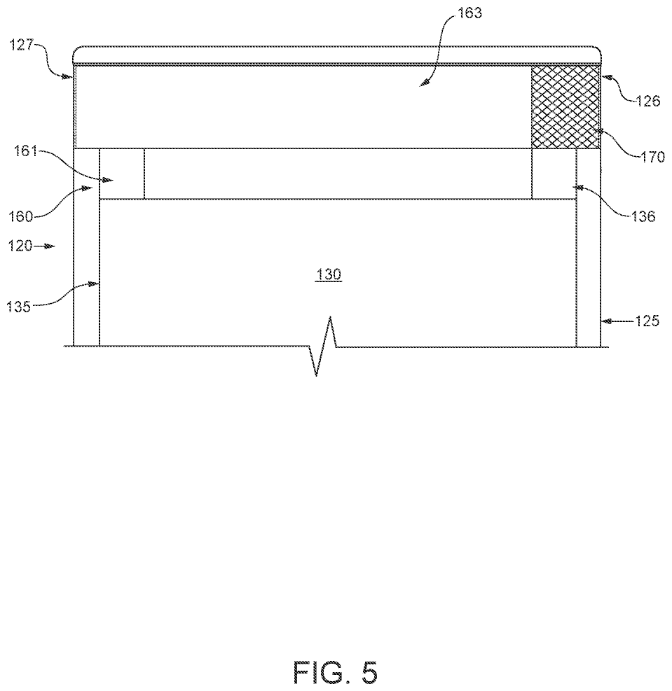

In some embodiments, the warming system 10 further includes a particulate air filtration unit 170 for removing particles from air passing therethrough. The particulate air filtration unit 170 is in fluid communication with the warming unit 120. As best shown in FIG. 5, the particulate air filtration unit 170 is located in the upper portion of the housing 125 and is fluidly connected to the air inlet 126 and to the upper circulation cavity 163. Furthermore, the particulate air filtration unit 170 is in fluid communication with the upper circulation unit 161 and the air outlet 127 via the upper circulation cavity 163, and with the warming compartment 130 via a recirculation vent 136.

In some embodiments, the particulate air filtration unit 170 is a high efficiency particulate air (HEPA) filtration unit.

In some implementations of these embodiments, the particulate air filtration unit 170 further includes an ultraviolet germicidal irradiation system which may, under certain circumstances, kill or inactivate some organic airborne contaminants such as bacteria, viruses, molds and other pathogens and prevent the transmission of diseases associated thereto.

In some embodiments, the particulate air filtration unit 170 is composed of a filter medium disposed within a frame, the filter medium having fibers arranged for intercepting airborne particles based on size, while allowing air to flow therethrough with minimal restriction. In some implementations of these embodiments, the particulate air filtration 170 unit removes at least 85% of particles, at least 90% of particulates, at least 91% of particulates, at least 92% of particulates, at least 93% of particulates, at least 94% of particulates, at least 95% of particulates, at least 96% of particulates, at least 97% of particulates, at least 98% of particulates, at least 99% of particulates. In some embodiments, the particulate air filtration 170 unit removes at least 99.5%, at least 99.6%, at least 99.7%, at least 99.8% or at least 99.9% of particulates.

In some embodiments, the particulates that are to be removed by the filtration unit 170 have a size that is at least 0.1 micron, at least 0.2 micron, at least 0.3 micron, at least 0.4 micron, at least 0.5 micron or at least 1.0 micron. In some instances, the particulate air filtration unit 170 is one of a semi-high efficiency particulate air (semi-HEPA) filtration unit, a high efficiency particulate air (HEPA) filtration unit and an ultra-low penetration air (ULPA) filtration unit.

In some embodiments, a circulation pathway 11 is defined between the air inlet 126, the particulate air filtration unit 170, the upper circulation cavity 163, and the air outlet 127. In some implementations, a recirculation pathway 12 is defined between the upper circulation unit 161, the warming compartment 130, the lower circulation unit 162, the lower circulation cavity 164, the heating unit 150, the warming compartment 130 and the recirculation vent 136 (FIG. 4).

Controller

The warming system 10 comprises a controller 190 for controlling temperature of the warming unit 120. The controller 190 is connected to a power supply (not shown) and controls the supply of power to all electrical elements of the warming system 10. In some embodiments, the controller 190 may also comprise a fan (not shown) for cooling the controller 190. As best shown on FIG. 1, the controller 190 includes an input device 195 external to the warming system 10 such that the input device 195 is manipulable by a user.

In some embodiments, the warming system 10 has at least one warming unit 120 having at least one warming compartment 130, at least one circulation unit 160 and a controller 190. The controller 190 is operatively connected to at least one warming unit 120 such that the desired temperature imparted to the at least one warming component 130 is selectably controllable and/or automatically controllable.

The controller 190 may also be used to operate the warming system 10 according to a plurality of operation modes. In some embodiments, the controller 190 includes sensors for detection of temperature (not shown) and sensors for detection of pressure (not shown) inside of the at least one warming compartment 130 and outside of the housing 125.

In some embodiments, the air inlet 126 includes an inlet valve (not shown), the air outlet 127 includes an outlet valve (not shown), the at least one circulation unit 160 includes a pump (not shown), and the controller 190 is operatively connected to the inlet valve, the outlet valve and the pump such that air flow and pressure inside the at least one warming compartment 130 are selectably adjustable and/or automatically adjustable.

In some embodiments, the controller 190 is operatively connected to the door 50, such that the door 50 may be locked automatically or selectably. The controller 190 may lock the door 50 in the closed position when the air pressure inside the at least one warming compartment 130 is above air pressure outside of the housing 125, when the air pressure inside the at least one warming compartment 130 is below air pressure outside of the housing 125, when temperature inside the at least one warming compartment 130 is above temperature outside of the housing 125, and when temperature inside the at least one warming compartment 130 is below temperature outside of the housing 125. In some implementations, the user may selectably lock the door 50 using the input device 195.

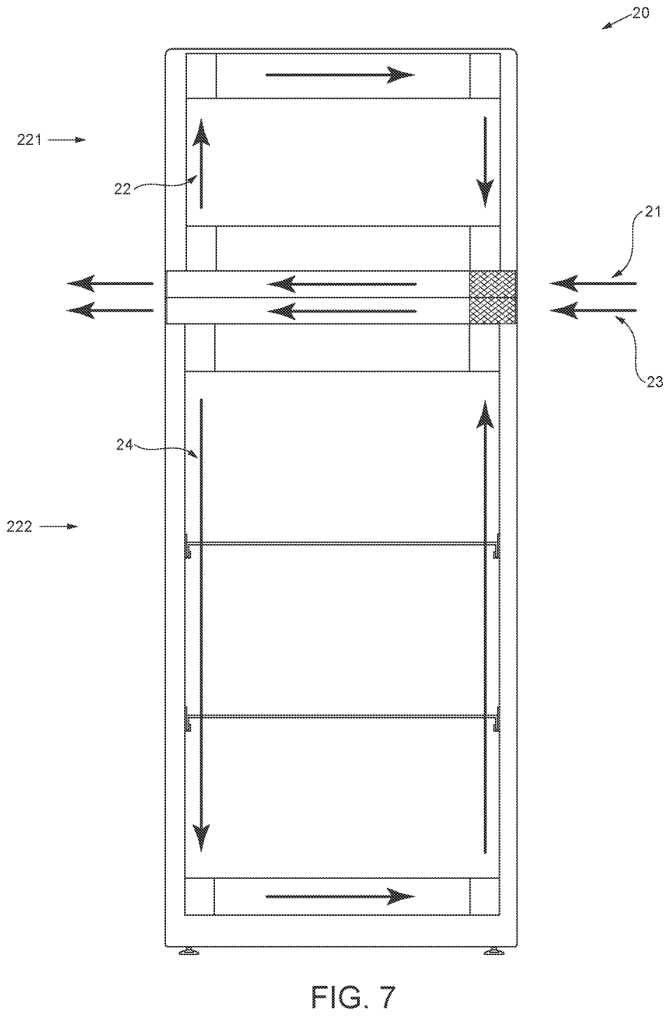

Referring to FIGS. 6 and 7, another embodiment of the present technology is shown. A warming system 20 comprises a housing 225 having first and second warming units 221, 222. In this embodiment, the first and second warming units 221, 222 are located in upper and lower portions of the housing 225, respectively, the first warming unit 221 being located above the second warming unit 222 and being vertically-aligned therewith. It is contemplated that in other embodiments, the first and second warming units 221, 222 may be located in different portions of the housing 225 and be aligned differently with one another.

The first and second warming units 221, 222 have first and second air inlets 226, 228, first and second air outlets 227, 229, first and second warming compartments 231, 232, first and second heating units 251, 252, first 262, 263 and second 264, 265 circulation units and first and second particulate air filtration units 271, 272. First air inlet 226, first warming compartment 231, first heating unit 251, first upper and lower circulation units 262, 263, first particulate air filtration unit 271 and first air outlet 227 are at least indirectly fluidly connected to one another. Second air inlet 228, second warming compartment 232, second heating unit 252, second upper and lower circulation units 264, 265, second particulate air filtration unit 272 and second air outlet 229 are at least indirectly fluidly connected to one another.

In some implementations, first air inlet 226, first air outlet 227, first heating unit 251, first circulation units 262, 263 and first door (not shown) are controlled by a controller 290 independently from second air inlet 228, second air outlet 229, second heating unit 252, second circulation units 264, 265 and second door (not shown), respectively. In some implementations, the warming units 221, 222 are not fluidly connected. In some implementations, the warming units 221, 222 are fluidly connected.

In the present embodiment, a first circulation pathway 21 is defined between the first air inlet 226, the first particulate air filtration unit 271, the first lower circulation cavity 273 and the first air outlet 227. A second circulation pathway 23 is defined between the second air inlet 228, the second particulate air filtration unit 272, the second upper circulation cavity 274 and the second air outlet 229.

In some implementations, a first recirculation pathway 22 is defined between the first heating unit 251, the first warming compartment 231, the first recirculation vent 275, the first upper circulation cavity 277, the first upper circulation unit 262, the first warming compartment 231 and the first lower circulation unit 263.

In some implementations, a second recirculation pathway 24 is defined between the second upper circulation unit 264, the second warming compartment 232, the second lower circulation unit 265, the second lower circulation cavity 276, the second heating unit 252, the second warming compartment 232, and the second recirculation vent 278 (FIG. 7).

In some aspects of the present technology, a warming system for heating medical items is adapted for use with a particulate air filtration unit for removing particles from air flowing therethrough. Such warming systems comprise a housing having an air inlet fluidly connectable to the particulate air filtration unit, at least one warming compartment and an air outlet. The warming compartment defines an access opening for receiving the medical items thereto, and the air inlet, the air outlet, the at least one warming compartment as well as the access opening are in fluid communication. The warming system further comprises a door and at least one heating unit. The door is structured and arranged with respect to the warming compartment such that the warming compartment is rendered accessible and in direct fluid communication with the clinical environment when the door is in an open position and is rendered inaccessible and not in direct fluid communication with the clinical environment when the door is in a closed position. The at least one heating unit is operatively connected to the at least one warming compartment for imparting a desired temperature thereto. The housing and the door are structured and arranged such that when the door is in a closed position, air flows through the particulate air filtration unit before flowing into the housing. Such warming systems may be adapted, under certain circumstances, for HVAC systems of clinical environments having a particulate air filtration unit that is structured and arranged such that the air inlet may be connected thereto.

It should be appreciated that the disclosure is not limited to the particular embodiments described and illustrated herein but includes all modifications and variations falling within the scope of the subject matters as defined in the appended claims.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A warming system for warming medical items comprising:
at least one warming unit for receiving the medical items, wherein the at least one warming unit maintains a desired temperature;
a circulation unit for circulating air through the at least one warming unit; and
a particulate air filtration unit for removing particles of air passing therethrough, the particulate air filtration unit in fluid communication with the at least one warming unit.

2. The warming system as defined in claim 1, wherein the particulate air filtration unit removes at least 85% of particles from air passing therethrough.

3. The warming system as defined in claim 1, wherein the particulate air filtration unit removes at least 90% of particles from air passing therethrough.

4. The warming system as defined in claim 1, wherein the particulate air filtration unit removes at least 95% of particles from air passing therethrough.

5. The warming system as defined in claim 1, wherein the particulate air filtration unit removes at least 99% of particles from air passing therethrough.

6. The warming system as defined in claim 1, wherein the particulate air filtration unit removes at least 99.9% of particles from air passing therethrough.

7. The warming system as defined in claim 1, wherein the particles have a size of at least about 1 micron.

8. The warming system as defined in claim 1, wherein the particles have a size of at least about 0.5 micron.

9. The warming system as defined claim 1, wherein the particles have a size of at least about 0.3 micron.

10. The warming system as defined in claim 1, wherein the particles have a size of at least about 0.1 micron.

11. The warming system according to claim 1, wherein the particulate air filtration unit is one of a semi-high efficiency particulate air (HEPA) filtration unit, a high efficiency particulate air (HEPA) filtration unit, and an ultra-low penetration air (ULPA) filtration unit.

12. The warming system according to claim 1, wherein the particulate air filtration unit is a high efficiency particulate air (HEPA) filtration unit.

13. The warming system according to claim 1, further comprising a controller for controlling the temperature of the at least one warming unit.

14. The warming system according to claim 13, wherein the controller comprises a fan for cooling the controller.

15. The warming system according to claim 14, the controller comprising an input device manipulable externally to the warming system by a user.

16. The warming system according to claim 1, wherein the at least one warming unit comprises a housing and a warming compartment disposed within said housing to receive the medical items.

17. The warming system according to claim 16, wherein the at least one warming unit further comprises a temperature sensor to measure temperature in the warming compartment.

18. The warming system according to claim 1, wherein the circulation unit comprises a hot air inlet and a hot air outlet in fluid communication with the at least one warming unit.

19. The warming system according to claim 18, wherein the hot air inlet is located in a lower portion of the warming system.

20. The warming system according to claim 18, wherein the hot air outlet is located in an upper portion of the warming system.
